(19) United States
(12) Patent Application Publication
(10) Pub. No.: US 2014/0102049 A1
(43) Pub. Date: Apr. 17, 2014

(54) Method Of Fabrication Of Implantable Medical Device Comprising Macrocyclic Triene Active Agent And Antioxidant

(71) Applicant: ABBOTT CARDIOVASCULAR SYSTEMS INC., Santa Clara, CA (US)

(72) Inventors: Stephen D. Pacetti, San Jose, CA (US); Ni Ding, San Jose, CA (US)

(73) Assignee: Abbott Cardiovascular Systems Inc., Santa Clara, CA (US)

(21) Appl. No.: 13/654,194

(22) Filed: Oct. 17, 2012

(51) Int. Cl. A61L 31/08

(52) U.S. Cl. CPC A61L 31/08
USPC 53/476; 427/2.1; 427/2.25

(57) ABSTRACT

This invention relates to methods of including an oxygen-sensitive macrocyclic triene on an implantable medical device wherein the device includes separate antioxidant-containing layers above, below or both above and below the drug reservoir layer containing the macrocyclic triene.

### FIELD

This invention relates to a method of mitigating the degradation of oxygen-sensitive macrocyclic triene active agents on implantable medical devices.

### BACKGROUND

Until the mid-1980s, the accepted treatment for coronary atherosclerosis, i.e., narrowing of the coronary artery(ies) was coronary by-pass surgery. While being quite effective and having evolved to a relatively high degree of safety for such an invasive procedure, by-pass surgery still involves potentially serious complications and generally results in an extended recovery period.

With the advent of percutaneous transluminal coronary angioplasty (PTCA) in 1977, the scene changed dramatically. Using catheter techniques originally developed for heart exploration, inflatable balloons were deployed to re-open occluded regions in arteries. The procedure was relatively non-invasive, took a short time compared to by-pass surgery and recovery time was minimal. However, PTCA brought with it its own problems including vasospasm, elastic recoil of the stretched arterial wall and restenosis, the re-clogging of the treated artery due to neointimal hyperplasia in the vicinity of the procedure, any of which could undo much of what was accomplished.

The next improvement, advanced in the mid-1980s, was the use of a stent to maintain a luminal diameter that had been re-established using PTCA. This for all intents and purposes put an end to vasospasm and elastic recoil but did not resolve the issue of restenosis. That is, prior to the introduction of stents, restenosis occurred in about 30 to 50% of patients undergoing PTCA. Stenting reduced this to about 15 to 20%, a substantial improvement but still more than desirable.

In 2003, the drug-eluting stent (DES) was introduced. The drugs initially used with DESs were cytostatic compounds, that is, compounds that curtailed the proliferation of cells that fostered restenosis. The occurrence of restenosis was reduced to about 5 to 7%, a relatively acceptable figure. However, the use of DESS engendered yet another complication, late stent thrombosis, the forming of blood clots at some time after the stent was in place. It was hypothesized that the formation of blood clots was most likely due to delayed healing, a side-effect of the use of cytostatic drugs. Thus, other types of drugs were sought to reduce the incidence of late stent thrombosis as well as other complications incident to the use of such agent. A promising solution was found in the anti-proliferative family of compounds, in particular rapamycin, a macrocyclic compound having in the ring structure three conjugated double bonds, and derivatives and analogs thereof, which appeared to be markedly effective. Thus, DESs including members of the rapamycin family of compounds were extensively studied, resulting in several becoming commercial products. It was found, however, that the conjugated triene functionality of the compounds rendered them sensitive to oxidative degradation. That is, oxygen in and around the device containing the macrocyclic triene, fostered the formation of radical species that in turn initiated auto-oxidation of the triene moiety. The response to this negative property of the compounds was relatively clear to those skilled in the art: include a pharmaceutically acceptable antioxidant among the substances included on a macrocyclic triene-containing DES.

While a number of antioxidants and techniques for their use to prevent oxidation of macrocyclic triene compounds on DESs have been described and in some cases implemented, alternative methods, which may also be improvements, are always valuable additions to the art. The instant invention provides such alternative methods.

### SUMMARY

Thus, an aspect of this invention is a method of fabricating an implantable medical device comprising an oxygen-sensitive macrocyclic triene active agent comprising:
disposing a drug reservoir layer comprising a therapeutically effective amount of the oxygen-sensitive macrocyclic triene active agent over at least a portion of an implantable medical device body;
disposing an antioxidant layer comprising a pharmaceutically acceptable antioxidant over, under or both over and under the drug reservoir layer.

In an aspect of this invention, a barrier layer is disposed between the each antioxidant layer and the drug reservoir layer wherein the barrier layer is substantially impenetrable to the pharmaceutically acceptable antioxidant.

In an aspect of this invention, the pharmaceutically acceptable antioxidant is selected from the group consisting of butylated hydroxytoluene (BHT), butylated hydroxyanisole, tert-butyl hydroquinone, quinone, (C1-C12)alkyl gallate, resveratrol, an antioxidant thiol, cysteine, N-acetylcysteine, bucillamine, glutathione, 7-hydroxyethylrutoside, carvedilol, vitamin C, vitamin E, α-tocopherol, α-tocopherol acetate, lycopene, a flavanoid, carotene and carotenoids.

In an aspect of this invention, the amount of pharmaceutically acceptable antioxidant in the antioxidant layer(s) is, independently in each antioxidant layer, about 0.05 percent to about 5.0 percent of the total amount of the macrocyclic triene active agent in the drug reservoir layer.

In an aspect of this invention,the amount of pharmaceutically acceptable antioxidant in the antioxidant layer(s) is, independently in each antioxidant layer, about 0.1 percent to about 0.5 percent of the total amount of the macrocyclic triene active agent in the drug reservoir layer.

In an aspect of this invention, the amount of pharmaceutically acceptable antioxidant in the antioxidant layer(s) is, independently in each antioxidant layer, about 0.2 percent of the total amount of the macrocyclic triene active agent in the drug reservoir layer.

In an aspect of this invention, the pharmaceutically acceptable antioxidant is butylated hydroxytoluene (BHT).

In an aspect of this invention, disposing the BHT antioxidant layer over the drug reservoir layer comprises contacting a topcoat layer of the implantable medical device with an atmosphere comprising BHT.

In an aspect of this invention, the atmosphere of BHT comprises sublimated BHT.

In an aspect of this invention, the atmosphere of BHT further comprises ethylene oxide and steam.

In an aspect of this invention, incorporating the BHT in the antioxidant layer comprises contacting the antioxidant layer with a stent crimping apparatus, an interior surface of which comprises heated, inwardly mobile wedges, each wedge having a surface that is forceably contacted with the stent surface to crimp it, the wedge surfaces being coated with BHT.

In an aspect of this invention, the antioxidant layer disposed under the drug reservoir layer comprises a primer layer.

In an aspect of this invention, the drug reservoir layer is disposed over the implantable medical device body in an inert atmosphere from a solution that has been de-oxygenated.

In an aspect of this invention,the oxygen-sensitive macrocyclic triene active agent is selected from the group consisting of rapamycin, a rapamycin derivative, sirolimus, zotarolimus, everolimus, temsirolimus, deforolimus, merilimus, myolimus and novolimus.

In an aspect of this invention, the oxygen-sensitive macrocyclic triene active agent is everolimus.

In an aspect of this invention, the method further comprising encasing the stent in a light-tight container for storage prior to implantation in a patient in need thereof.

In an aspect of this invention, the implantable medical device comprises a stent.

### DETAILED DESCRIPTION

Brief description of the figures

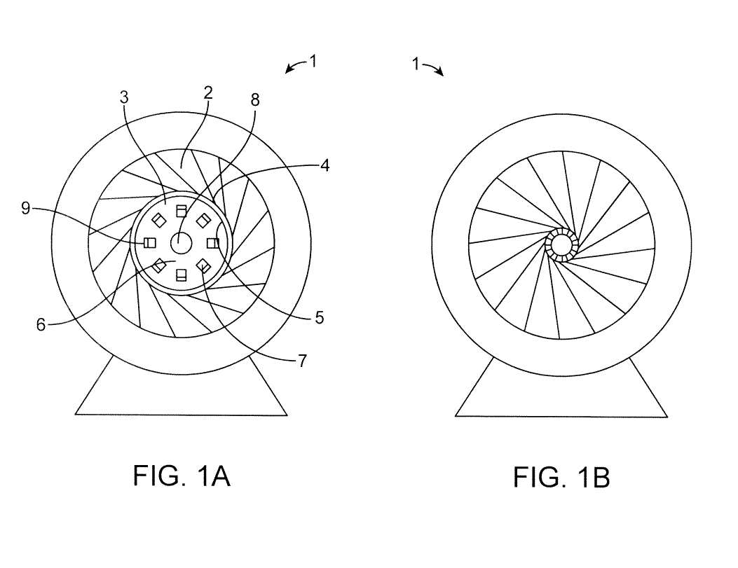

FIG. 1 shows a stent crimping device which may be used to apply an antioxidant to the stent.

FIG. 1A shows the crimping device in its expanded state before the stent is crimped to a delivery catheter.

FIG. 1B shows the crimping device in its contracted state after the stent has been crimped onto the delivery device.

### DISCUSSION

Use of the singular herein includes the plural and vice versa unless expressly stated to be otherwise. That is, “a” and “the” refer to one or more of whatever the word modifies. For example, “a pharmaceutically acceptable antioxidant” includes one such oxidant, two such oxidants or, under the right circumstances, an even greater number of antioxidants. Likewise, “the layer” may refer to one, two or more layers and “the polymer” may mean one polymer or a plurality of polymers. By the same token, words such as, without limitation, “layers” and “polymers” refer to one layer of polymer as well as to a plurality of layers or polymers unless, again, it is expressly stated or obvious from the context that such is not intended.

As used herein, words of approximation such as, without limitation, “about” “substantially,” “essentially” and “approximately” mean that the feature so modified need not be exactly that which is expressly described but may vary from that written description to some extent. The extent to which the description may vary will depend on how great a change can be instituted and have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±15%.

As used herein, an “implantable medical device” refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device is physically removed; or until the device biodegrades usually as the intentional use of a biodegradable substance for the fabrication of the device such that the device degrades over a predetermined time-span. Examples of implantable medical devices include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, balloon-expandable stents, stent-grafts, grafts, artificial heart valves and cerebrospinal fluid shunts.

While implantable medical devices can serve several concurrent purposes and such are within the scope of this invention, an implantable medical device specifically designed and intended solely for the localized delivery of a therapeutic agent is within the scope of this invention.

Presently preferred implantable medical devices of this invention are stents.

A stent refers generally to a device used to hold tissue in place in a patient's body. Particularly useful stents, however, are those used for the maintenance of the patency of a vessel in a patient's body when the vessel is narrowed or closed due to diseases or disorders including, without limitation, tumors (in, for example, bile ducts, the esophagus, the trachea/bronchi, etc.), benign pancreatic disease, coronary artery disease, carotid artery disease and peripheral arterial disease such as atherosclerosis, restenosis and vulnerable plaque. Vulnerable plaque (VP) refers to a fatty build-up in an arterial wall thought to be caused by inflammation. The VP is covered by a thin fibrous cap that can rupture leading to blood clot formation. A stent can be used to strengthen the wall of the vessel in the vicinity of the VP and act as a shield against such rupture. A stent can be used in, without limitation, neuro, carotid, coronary, pulmonary, aorta, renal, biliary, iliac, femoral and popliteal as well as other peripheral vasculatures. A stent can be used in the treatment or prevention of disorders such as, without limitation, thrombosis, restenosis, hemorrhage, vascular dissection or perforation, vascular aneurysm, chronic total occlusion, claudication, anastomotic proliferation, bile duct obstruction and ureter obstruction.

In addition to the above uses, stents may also be employed for the localized delivery of therapeutic agents to specific treatment sites in a patient's body. In fact, therapeutic agent delivery may be the sole purpose of the stent or the stent may be primarily intended for another use such as those discussed above with drug delivery providing an ancillary benefit.

A stent used for patency maintenance is usually delivered to the target site in a compressed state and then expanded to fit the vessel into which it has been inserted. Once at a target location, a stent may be self-expandable or balloon expandable. In any event, due to the expansion of the stent, any coating thereon must be flexible and capable of elongation.

As used herein, “device body” refers to a fully formed implantable medical with an outer surface to which no coating or layer of material different from that of which the device itself is manufactured has been applied. A common example of a device body is a bare metal stent (BMS), which, as the name implies, is a fully-formed, usable stent that has not been coated with a layer of any material different from the metal of which it is made on any surface that is in contact with bodily tissue or fluids. Of course, device body refers not only to BMSs but to any uncoated device regardless of what it is made of.

Implantable medical devices made of virtually any material, i.e., materials presently known to be useful for the manufacture of implantable medical devices and materials that may be found to be so in the future, may be used in the method of this invention. For example, without limitation, an implantable medical device useful with this invention may be made of one or more biocompatible metals or alloys thereof including, but not limited to, cobalt-chromium alloy (ELGILOY, L-605), cobalt-nickel alloy (MP-35N), 316L stainless steel, high nitrogen stainless steel, e.g., BIODUR 108, nickel-titanium alloy (NITINOL), tantalum, platinum, platinum-iridium alloy, gold and combinations thereof.

Implantable medical devices may also be made of polymers that are biocompatible and biostable or biodegradable, the latter term including bioabsorbable and/or bioerodable.

As used herein, “biocompatible” refers to a polymer that both in its intact as synthesized state and in its decomposed state, i.e., its degradation products, is not, or at least is minimally toxic to living tissue; does not, or at least minimally and reparably injures living tissue; and/or does not, or at least minimally and/or controllably causes an immunological reaction in living tissue. Biocompatible polymers of this invention may be biostable or biodegradable where “biodegradable” simply means that the polymer will be decomposed over time when exposed to a physiological environs, i.e. to the conditions present in a patient's body such as pH, the presence of enzymes, body temperature, etc.

Examples of biocompatible, relatively biostable polymers that may be used with an implantable medical device of this invention include, without limitation, polyacrylates, polymethacryates, polyureas, polyurethanes, polyolefins, polyvinylhalides, polyvinylidenehalides, polyvinylethers, polyvinylaromatics, polyvinylesters, polyacrylonitriles, polysiloxanes, alkyd resins and epoxy resins.

Biocompatible, biodegradable polymers include naturally-occurring polymers such as, without limitation, collagen, chitosan, alginate, fibrin, fibrinogen, cellulosics, starches, dextran, dextrin, hyaluronic acid, heparin, glycosaminoglycans, polysaccharides and elastin.

One or more synthetic or semi-synthetic biocompatible, biodegradable polymers may also be used to fabricate an implantable medical device body useful with this invention. As used herein, a synthetic polymer refers to one that is created wholly in the laboratory while a semi-synthetic polymer refers to a naturally-occurring polymer that has been chemically modified in the laboratory. Examples of synthetic polymers include, without limitation, polyphosphazines, polyphosphoesters, polyphosphoester urethane, polyhydroxyacids, polyhydroxyalkanoates, polyanhydrides, polyesters, polyorthoesters, polyamino acids, polyoxymethylenes, poly(ester-amides) and polyimides.

Other biocompatible biodegradable polymers that may be used with the device and method of this invention include, without limitations, polyesters, polyhydroxyalkanoates (PHAs), poly(ester amides) that may optionally contain alkyl, amino acid, PEG and/or alcohol groups, polycaprolactone, poly(L-lactide), poly(D,L-lactide), poly(D,L-lactide-co-PEG) block copolymers, poly(D,L-lactide-co-trimethylene carbonate), polyglycolide, poly(lactide-co-glycolide), polydioxanone (PDS), polyorthoester, polyanhydride, poly(glycolic acid-co-trimethylene carbonate), polyphosphoester, polyphosphoester urethane, poly(amino acids), polycyanoacrylates, poly(trimethylene carbonate), poly(iminocarbonate), polycarbonates, polyurethanes, copoly(ether-esters) (e.g. PEO/PLA), polyalkylene oxalates, polyphosphazenes, PHA-PEG, and combinations thereof. The PHA may include poly(α-hydroxyacids), poly(β-hydroxyacid) such as poly(3-hydroxybutyrate) (PHB), poly(3-hydroxybutyrate-co-valerate) (PHBV), poly(3-hydroxyproprionate) (PHP), poly(3-hydroxyhexanoate) (PHH), or poly(4-hydroxyacid) such as poly poly(4-hydroxybutyrate), poly(4-hydroxyvalerate), poly(4-hydroxyhexanoate), poly(hydroxyvalerate), poly(tyrosine carbonates), poly(tyrosine arylates), poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide-co-lactic acid) (PEO/PLA)), polyalkylene oxides such as poly(ethylene oxide), poly(propylene oxide), poly(ether ester), polyalkylene oxalates, phosphoryl choline containing polymer, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, methacrylate polymers containing 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, elastin, elastin protein mimetics, or combinations thereof. Some examples of elastin protein mimetics include (LGGVG)ₙ, (VPGVG)ₙ, Val-Pro-Gly-Val-Gly, or synthetic biomimetic poly(L-glytanmate)-b-poly(2-acryloyloxyethyllactoside)-b-poly(l-glutamate) triblock copolymer.

In some embodiments of the current invention the polymer used with the device and in the method of this invention can be poly(ethylene-co-vinyl alcohol), poly(methoxyethyl methacrylate), poly(dihydroxylpropyl methacrylate), polymethacrylamide, aliphatic polyurethane, aromatic polyurethane, nitrocellulose, poly(ester amide benzyl), co-poly-{[N,N′-sebacoyl-bis-(L-leucine)-1,6-hexylene diester]_{0.75}-[N,N′-sebacoyl-L-lysine benzyl ester]_{0.25}} (PEA-Bz), co-poly-{[N,N′-sebacoyl-bis-(L-leucine)-1,6-hexylene diester]_{0.75}-[N,N′-sebacoyl-L-lysine-4-amino-TEMPO amide]_{0.25}} (PEA-TEMPO), aliphatic polyester, aromatic polyester, fluorinated polymers such as poly(vinylidene fluoride-co-hexafluoropropylene), poly(vinylidene fluoride) (PVDF), and Teflon™ (polytetrafluoroethylene), a biopolymer such as elastin mimetic protein polymer, star or hyper-branched SIBS (styrene-block-isobutylene-block-styrene), or combinations thereof. In some embodiments, where the polymer is a copolymer, it can be a block copolymer that can be, e.g., di-, tri-, tetra-, or oligo block copolymers or a random copolymer. In some embodiments, the polymer can also be branched polymers such as star polymers.

Presently preferred polymers for use with this invention include polyesters such as, without limitation, poly(L-lactide), poly(D-lactide), poly(D,L-lactide), poly(meso-lactide), poly(L-lactide-co-glycolide), poly(D-lactide-co-glycolide), poly (D,L-lactide-co-glycolide), poly(meso-lactide-co-glycolide), poly(caprolactone), poly(hydroxyvalerate), poly(hydroxybutyrate), poly(ethylene glycol-co-butylene terephthalate).

Other presently preferred polymers of this invention are fluoropolymers such as poly(vinylidene fluoride-co-hexafluoropropylene). When used, the poly(vinylidene fluoride-co-hexafluoropropylene) preferable at present has a constitutional unit weight-to-weight (wt/wt) ratio of about 85:15. “Constitutional unit” refers to the composition of a monomer as it appears in a polymer. For example, without limitation, the constitutional unit of the monomer acrylic acid, CH₂═CHC(O)OH, is —CH₂—CH₂C(O)O—. The average molecular weight of the presently preferred poly(vinylidene fluoride-co-hexafluoropropylene) polymer is from about 50,000 to about 500,000 Daltons. Further, it is presently preferred that the poly(vinylidene fluoride-co-hexafluoropropylene) polymer used to form a drug reservoir layer herein be semicrystalline. The presently preferred coating thickness of the poly(vinylidene fluoride-co-hexafluoropropylene) drug reservoir layer is from about 1 um to about 20 um.

Blends and copolymers of the above polymers may also be used and are within the scope of this invention. Based on the disclosures herein, those skilled in the art will recognize those implantable medical devices and those materials from which they may be fabricated that will be useful with the coatings of this invention.

As used herein, a “primer layer” refers to a coating consisting of a polymer or blend of polymers that exhibit good adhesion characteristics with regard to the material of which the device body is manufactured and good adhesion characteristic with regard to whatever material is to be coated on the device body. Thus, a primer layer serves as an intermediary layer between a device body and materials to be affixed to the device body and is, therefore, applied directly to the device body. Examples without limitation, of primers include acrylate and methacrylate polymers with poly(n-butyl methacrylate) being a presently preferred primer. Some additional examples of primers include, but are not limited to, poly(ethylene-co-vinyl alcohol), poly(vinyl acetate-co-vinyl alcohol), poly(methacrylates), poly(acrylates), polyethyleneamine, polyallylamine, chitosan, poly(ethylene-co-vinyl acetate), and parylene-C.

As use herein, a material that is described as a layer “disposed over” an indicated substrate be it a device body or another layer, refers to a coating of the material applied directly to the exposed surface of the indicated substrate. By “exposed surface” is meant any surface regardless of its physical location with respect to the configuration of the device that, in use, would be in contact with bodily tissues or fluids. “Disposed over” may, however, also refer to the application of the layer onto an intervening layer that has been applied to a stent body, wherein the layer is applied in such a manner that, were the intervening layer not present, the layer would be applied to the exposed surface of the indicated substrate. An example of an intervening layer is a primer layer.

Disposing “over” or “under” a drug reservoir layer is referenced to, as would be expected, the external environment. That is, disposing an antioxidant layer under a drug reservoir layer means that the drug reservoir layer is between the antioxidant layer and the external environment. Conversely, disposing an antioxidant layer over a drug reservoir layer means that the antioxidant layer is between the drug reservoir layer and the external environment.

As used herein, an “antioxidant layer” refers to a separate layer of material that includes a pharmaceutically acceptable antioxidant and may include additional substances except for the oxygen sensitive macrocyclic triene active agent, of which there is initially none in the antioxidant layer. By “initially none” is meant that, at least at the time of application of an antioxidant layer to an implantable medical device, there is no macrocyclic triene in the composition being applied that contains an antioxidant.

As used herein, “drug reservoir layer” refers either to a layer of therapeutic agent applied neat applied as a layer comprising a polymer that has dispersed within its three-dimensional structure the therapeutic agent. A polymeric drug reservoir layer is designed such that, by one mechanism or another, e.g., without limitation, by elution or as the result of biodegradation of the polymer, the therapeutic substance is released from the layer into the surrounding environment. A drug reservoir layer may also act as rate-controlling layer. Conversely to the situation above regarding the antioxidant layer, a drug reservoir layer contains initially none of the antioxidant. As above, by “initially none, is meant that at the time of application of the drug reservoir layer to an implantable medical device, there is no antioxidant in the composition being applied that contains the macrocyclic triene.

As used herein, a “topcoat layer” refers to a polymeric layer that is disposed over an implantable medical device of this invention such that it comprises the outermost layer of polymer on the device, that is, it is the layer that is in direct contact with the environment in which the device implanted. A topcoat layer is generally biodegradable, which biodegradation may occur relatively slowly if the layer is also serves as a rate controlling layer for the release of the macrocyclic triene from the device, or biodegradation may occur rapidly if the topcoat layer serves only as a protective layer for the layers underneath. A topcoat layer may also serve as a compatibility-inducing layer that renders the device more inert with regard to reaction with foreign body-eliminating mechanisms with the body.

As used herein, a “barrier layer” refers to a polymeric layer that is substantially impermeable to one or more substances that might otherwise migrate to an adjoining layer were it not for the intervening barrier layer. For the purposes of this invention, a barrier layer would be impermeable to the antioxidants used to protect the macrocyclic triene from degradation. The barrier layer may be biostable or it may be biodegradable. A biostable barrier layer remains intact and impermeable to the selected substances for essentially the lifespan of an implantable medical device of which it is a part. A biodegradable barrier layer will decompose under the influence of the physiological environs encountered by the exposed surfaces of an implantable medical device once implanted, which physiological environs may include, but is not limited to higher temperatures, acidic or basic pH and functional group specific enzymes, that is, enzymes that dissemble certain function groups such that groups linked together by the functional groups come apart. The use of a barrier layer between a drug reservoir layer and an antioxidant layer or layers that may be disposed above or below it, is optional and may be employed if there is a desire to keep the macrocyclic triene and the protective antioxidant physically apart from one another.

As used herein a pharmaceutically acceptable antioxidant refers to a chemical substance that does not detrimentally affect the physiological well-being of a patient to whom the antioxidant has been administered and that is capable of preventing damage to therapeutic agents due to reaction of the agent with oxygen or free radicals released by reaction of oxygen with other entities. For the purpose of this invention, a pharmaceutically acceptable antioxidant may be taken from the group consisting of butylated hydroxytoluene, butylated hydroxyanisole, tert-butyl hydroquinone, quinone, (C1-C12)alkyl gallate, resveratrol, an antioxidant thiol, cysteine, N-acetylcysteine, bucillamine, glutathione, 7-hydroxyethylrutoside, carvedilol, vitamin C, vitamin E, α-tocopherol, α-tocopherol acetate, lycopene, a flavanoid, carotene and carotenoids.

A presently preferred antioxidant for use in the methods herein is butylated hydroxytoluene (BHT).

An antioxidant of this invention may be included on an implantable medical device in an amount that totals about 0.01 to about 5.0% of the total amount of macrocyclic triene active agent. Preferably at present the total amount of antioxidant is from about 0.1 to about 1.0% of the total amount of the macrocyclic triene associated with the implantable medical device. Most preferable at present, the total amount of antioxidant is about 0.2% of the total amount of macrocyclic triene associated with the device.

As used herein, the “atmosphere” in which an implantable medical device of this invention has disposed on it a macrocyclic triene active agent refers to the gaseous environment in which the deposition takes place. For the purpose of this invention, the atmosphere should be “inert,” that is, it is itself unreactive with a macrocyclic triene active agent of this invention and it should contain no other substance, such as oxygen, hat could react with the macrocyclic triene. Thus, an atmosphere of this invention may comprise, without limitation, nitrogen, argon, carbon dioxide, ethylene oxide and the like. While the overall environment may be regarded as “gaseous,” it is permissible and is an embodiment of this invention that the “atmosphere” may contain atomized particulate matter such as, without limitation, sublimated BHT and steam. In particular, the atmosphere may comprise ethylene oxide and steam, which serve to sterilize the entire system, along with sublimated BHT.

A solution that has been “deoxygenated” has been treated so as to remove substantially all dissolved oxygen. Such treatment may involve, without limitation, sparging with an inert gas such as nitrogen or argon, heating, preferably to a boil, or placing the solution under vacuum, optionally with cooling.

As used herein, “therapeutic agent” refers to any substance that, when administered in a therapeutically effective amount to a patient suffering from a disease, has a therapeutic beneficial effect on the health and well-being of the patient. A therapeutic beneficial effect on the health and well-being of a patient includes, but it not limited to: (1) curing the disease; (2) slowing the progress of the disease; (3) causing the disease to retrogress; or, (4) alleviating one or more symptoms of the disease. As used herein, a therapeutic agent also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease in the first place; (2) maintaining a disease at a retrogressed level once such level has been achieved by a therapeutically effective amount of a substance, which may be the same as or different from the substance used in a prophylactically effective amount; or, (3) preventing or delaying recurrence of the disease after a course of treatment with a therapeutically effective amount of a substance, which may be the same as or different from the substance used in a prophylactically effective amount, has concluded.

A “therapeutically effective amount” refers to that amount of a therapeutic agent that will have a beneficial effect, which may be curative or palliative, on the health and well-being of the patient with regard to the disease or disorder with which the patient is known or suspected to be afflicted. A therapeutically effective amount may be administered as a single bolus, as intermittent bolus charges, as short, medium or long term sustained release formulations or as any combination of these. As used herein, short-term sustained release refers to the administration of a therapeutically effective amount of a therapeutic agent over a period from about several hours to about 3 days. Medium-term sustained release refers to administration of a therapeutically effective amount of a therapeutic agent over a period from about 3 day to about 14 days and long-term refers to the delivery of a therapeutically effective amount over any period in excess of about 14 days. Any reference a therapeutic agent relating to its presence on an implantable medical device or its use in a method of this invention is to be understood as referring to a therapeutically effective amount of that therapeutic agent.

Presently preferred therapeutic agents of this invention are the macrocyclic trienes. As used herein, a “macrocyclic triene” refers generally to a compound having a ring structure that contains 12 or more atoms, and that includes at least three conjugated double bonds in the rings system. In particular, “macrocyclic triene” refers to rapamycin and derivatives and analogs thereof, including, at present, rapamycin itself, commonly known as sirolimus, zotarolimus, everolimus, temsirolimus, deforolimus, myolimus and novolimus. These compounds are “active agents” as set forth herein in that they are all mTOR inhibitors useful in the treatment of patients with damaged endothelia such as that which generally accompanies treatments such as PTCA, percutaneous transluminal angioplasty, for vascular disease. The term “therapeutic agent” is synonymous with “active agent,” and the two are interchangeable for the purposes of this disclose and attendant claims.

Presently preferred from among the macrocyclic trienes if everolimus.

The rapamycin macrocyclic trienes are oxygen sensitive due to the presence of the conjugated triene, i.e., three double bonds linked together by a single bond between the first and the second and a single bond between the second and the third. They are referred to herein as “oxygen sensitive macrocyclic triene active agents.” Since it is desirable, if not essential, that the composition and quantity of an active agent being administered to a patient, regardless of the manner of administration, be accurately known, it is highly desirable to control as well as possible any mechanism that might detrimentally affect the active agent before it is administered. Oxidation of compounds often has such a detrimental effect on active agents and is to be controlled. With regard to delivery of macrocyclic triene active agents of this invention using implantable medical devices such as stents, a solution to this problem lies in the inclusion of pharmaceutically acceptable antioxidant compounds on the device. By “pharmaceutically acceptable” is meant that the antioxidants that are useful in this invention have been found acceptable for use in humans by the Food and Drug Administration (FDA, in the United States; equivalent foreign governmental agencies would be charged with such approvals in their respective countries). Of course, antioxidants that may in the future be found acceptable for human use by the FDA are clearly within the scope of this invention. Antioxidants curtail oxidation of macrocyclic trienes by several well-known mechanisms such as radical scavenging and complexation with pro-oxidation metal species. BHT, a presently preferred antioxidant for use with an implantable medical device of this invention, is of the former type, i.e., it functions as a radical scavenger.

If desired it is entirely possible, and in fact is an aspect of this invention, to include another therapeutic agent or agents along with the macrocyclic triene on an implantable medical device hereof. If the other agent(s) are known to not be oxygen sensitive, then no changes need be made to the disclosure herein of the amount of antioxidant to use. If, on the other hand, any of the additional therapeutic agents are known to be oxygen sensitive, then the total amount of antioxidant used may be determined as set forth above except that the total amount of macrocyclic triene plus the amount of any other oxygen sensitive therapeutic agent(s) is used in the calculation.

Among other therapeutic agents that may be suitable for use in this invention, anti-inflammatory compounds are particularly presently preferred. Suitable anti-inflammatory agents that can be used in combination with the macrocyclic trienes herein include, without limitation, clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus and prodrugs, co-drugs and combinations thereof.

Other therapeutic agents that may be suitable for use in the methods herein include anti-neoplastic, antimitotic, antiplatelet, antifebrin, antithrombin, cytostatic and anti-proliferative agents.

Antineoplastic or anti-mitotic agents include, without limitation, paclitaxel, docetaxel, methotrexate, azathioprine, vincristine, vinblastine, fluorouracil, doxorubicin hydrochloride, and mitomycin.

Antiplatelet, anticoagulant, antifibrin, and antithrombin agents include, without limitation, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin and thrombin, thrombin inhibitors such as Angiomax ä, calcium channel blockers such as nifedipine, colchicine, fish oil (omega 3-fatty acid), histamine antagonists, lovastatin, monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO) and estradiol.

Cytostatic or anti-proliferative agents include, without limitation, angiopeptin, angiotensin converting enzyme inhibitors such as captopril, cilazapril or lisinopril, calcium channel blockers such as nifedipine; colchicine, fibroblast growth factor (FGF) antagonists; fish oil (ω-3-fatty acid); histamine antagonists; lovastatin, monoclonal antibodies such as, without limitation, those specific for Platelet-Derived Growth Factor (PDGF) receptors; nitroprusside, phosphodiesterase inhibitors, prostaglandin inhibitors, suramin, serotonin blockers, steroids, thioprotease inhibitors, triazolopyrimidine (a PDGF antagonist) and nitric oxide.

Other potentially useful therapeutic agents include, without limitation, alpha-interferon, genetically engineered epithelial cells, DNA and RNA nucleic acid sequences, antisense molecules, and ribozymes, antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides, retroviral vectors; antiviral agents; analgesics; anorexics; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers, beta-blockers such as pindolol, antiarrhythmics; antihypertensives; diuretics; vasodilators including general coronary; peripheral and cerebral; central nervous system stimulants; cough and cold preparations, including decongestants; hypnotics; immunosuppressives; muscle relaxants; parasympatholytics; psychostimulants; sedatives; tranquilizers; natural or genetically engineered lipoproteins; and restenosis reducing agents.

A presently preferred combination of elements of this invention includes a stent as the implantable medical device, everolimus as the macrocyclic triene and BHT as the antioxidant. The quantity of everolimus on a stent may vary and such variations are well-known to those skilled in the art based a several commercial everolimus-containing stents. With regard to the BHT, it is preferably present on an everolimus stent in a total amount that is about 0.1% to about 0.5%, most preferably about 0.2%, of the total amount of everolimus on a stent. The BHT may be incorporated on the stent in several ways. For example, without limitation, the BHT may simply be included, by dissolution or suspension in a solvent in which a polymer used to form any layer other than an everolimus-containing drug reservoir layer is contained. This could be, for instance, a primer layer, a topcoat layer, protective layer or any combination thereof. The BHT may alternatively be infiltration into any layer of the stent, again, other than a layer that includes everolimus, from an atmosphere that includes sublimated BHT. Generally, if a BHT atmosphere is being used, the layer into which the BHT infiltrates is preferable an exposed outer layer on a device herein. Such layer may be a topcoat layer, a protective layer or any other layer other than the drug reservoir layer itself. As described above, the use of sublimated BHT comprises exposing a layer of the stent to an atmosphere of sublimated BHT alone or to an atmosphere containing sublimated BHT, ethylene oxide and steam, the latter two substances being present to sterilize the stent.

A further technique for forming an antioxidant layer on a stent is to use a stent crimping apparatus such as that shown in FIG. 1. In FIG. 1, sliding wedge crimper 1 comprises slideable crimping wedges 2 and a central lumen 3 defined by internal surfaces 4 of slideable wedges 2. Internal surfaces 4 of crimping wedges 2 are coated with the desired antioxidant, 5. The coating may be applied by any means known to those skilled in the art, the simplest of which is to apply solvent containing the antioxidant to the surface of wedges 2 under conditions that permit the rapid evaporation of the solvent, which may include, without limitation, heating the wedges or the general environment in which the application is carried out. A delivery catheter, 8, which has been inserted into central lumen 6 of stent 7, shown in cross-section, is inserted into lumen 3. When slideable wedges 2 are rotated, they reduce the diameter of central lumen 3 resulting in the crimping of stent 7 onto the delivery catheter. As a result, antioxidant 5 is transferred onto stent 7, either neat or into a layer of material 9, which has been previously coated onto stent 7. Pressure alone may be the driving force for transfer of antioxidant 5 to stent 7 or crimping wedges 2 may be heated to facilitate the transfer.

The method herein may also include enclosing the finished implantable medical device in a light-tight container, that is, a container through which at least visible and ultraviolet light cannot penetrate. Thus the antioxidant essentially surrounding the drug reservoir layer will protect the macrocyclic triene from radicals formed due to the presence of oxygen or other radical species attempting to penetrate into the drug reservoir layer from the surrounding layers, the use of the light-tight container will prevent radicals from potentially being formed directed in the drug reservoir layer by the interaction of light with light sensitive radical forming species.

What is claimed:

1. A method of fabricating an implantable medical device comprising an oxygen-sensitive macrocyclic triene active agent comprising:
disposing a drug reservoir layer comprising a therapeutically effective amount of the oxygen-sensitive macrocyclic triene active agent over at least a portion of an implantable medical device body;
disposing an antioxidant layer comprising a pharmaceutically acceptable antioxidant over, under or both over and under the drug reservoir layer.

2. The method of claim 1, wherein a barrier layer is disposed between the each antioxidant layer and the drug reservoir layer wherein the barrier layer is substantially impenetrable to the pharmaceutically acceptable antioxidant.

3. The method of claim 1, wherein the pharmaceutically acceptable antioxidant is selected from the group consisting of butylated hydroxytoluene (BHT), butylated hydroxyanisole, tert-butyl hydroquinone, quinone, (C1-C12)alkyl gallate, resveratrol, an antioxidant thiol, cysteine, N-acetylcysteine, bucillamine, glutathione, 7-hydroxyethylrutoside, carvedilol, vitamin C, vitamin E, α-tocopherol, α-tocopherol acetate, lycopene, a flavanoid, carotene and carotenoids.

4. The method of claim 3, where the amount of pharmaceutically acceptable antioxidant in the antioxidant layer(s) is, independently in each antioxidant layer, about 0.05 percent to about 5.0 percent of the total amount of the macrocyclic triene active agent in the drug reservoir layer.

5. The method of claim 3, wherein the amount of pharmaceutically acceptable antioxidant in the antioxidant layer(s) is, independently in each antioxidant layer, about 0.1 percent to about 0.5 percent of the total amount of the macrocyclic triene active agent in the drug reservoir layer.

6. The method of claim 3, wherein the amount of pharmaceutically acceptable antioxidant in the antioxidant layer(s) is, independently in each antioxidant layer, about 0.2 percent of the total amount of the macrocyclic triene active agent in the drug reservoir layer.

7. The method of claim 3, wherein the pharmaceutically acceptable antioxidant is butylated hydroxytoluene (BHT).

8. The method of claim 7, wherein disposing the BHT antioxidant layer over the drug reservoir layer comprises contacting a topcoat layer of the implantable medical device with an atmosphere comprising BHT.

9. The method of claim 8, wherein the atmosphere of BHT comprises sublimated BHT.

10. The method of claim 8, wherein the atmosphere of BHT further comprises ethylene oxide and steam.

11. The method of claim 7, wherein the implantable medical device comprises a stent.

12. The method of claim 7, wherein incorporating the BHT in the antioxidant layer comprises contacting the antioxidant layer with a stent crimping apparatus, an interior surface of which comprises heated, inwardly mobile wedges, each wedge having a surface that is forceably contacted with the stent surface to crimp it, the wedge surfaces being coated with BHT.

13. The method of claim 1, wherein the antioxidant layer disposed under the drug reservoir layer comprises a primer layer.

14. The method of claim 1, wherein the drug reservoir layer is disposed over the implantable medical device body in an inert atmosphere from a solution that has been de-oxygenated.

15. The method of claim 1, wherein the oxygen-sensitive macrocyclic triene active agent is selected from the group consisting of rapamycin, a rapamycin derivative, sirolimus, zotarolimus, everolimus, temsirolimus, deforolimus, merilimus, myolimus and novolimus.

16. The method of claim 7, wherein the oxygen-sensitive macrocyclic triene active agent is everolimus.

17. The method of claim 1, further comprising encasing the stent in a light-tight container for storage prior to implantation in a patient in need thereof.
